(19) United States
(12) Patent Application Publication
(10) Pub. No.: US 2020/0179308 A1
(43) Pub. Date: Jun. 11, 2020

(54) USE OF ISOHUMULONES AND DERIVATIVES THEREOF TO TREAT POLYCYSTIC OVARY SYNDROME

(71) Applicant: KINDEX PHARMACEUTICALS, INC., Seattle, WA (US)

(72) Inventors: Jeffrey BLAND, Seattle, WA (US); Neile GRAYSON, Seattle, WA (US); Andrew WOLFE, Baltimore, MD (US); Sheng WU, Baltimore, MD (US)

(21) Appl. No.: 16/604,533

(22) PCT Filed: Apr. 12, 2018
(86) PCT No.: PCT/US2018/027339

§ 371 Date: Oct. 10, 2019

Related U.S. Application Data
(60) Provisional application No. 62/484,835, filed on Apr. 12, 2017.

(51) Int. Cl. A61K 31/122; A61P 15/08

(52) U.S. Cl. CPC A61K 31/122; A61P 15/08

(57) ABSTRACT

The present disclosure provides methods of treating polycystic ovary syndrome (PCOS) with tetrahydro-iso-alpha acid (THIAA) derivatives and substantially enantiomerically pure compositions and pharmaceutical formulations thereof, in particular KDT500 and KDT501.

### PRIORITY CLAIM

This application claims priority to U.S. Provisional Application No. 62/484,835, filed Apr. 12, 2017, the disclosure of which is incorporated by reference herein in its entirety.

### BACKGROUND

Female reproductive function is acutely sensitive to changes in the external environment, but is also exquisitely responsive to changes in the internal hormonal and metabolic milieu. Modest changes in the timing or magnitude of the dynamic hormone changes can disrupt female reproductive function. Polycystic ovary syndrome (PCOS) is a complex, heterogeneous disorder impacting approximately 5-21% of pre-menopausal women. PCOS is characterized by hyperandrogenism and anovulation/oligo-ovulation and is frequently associated with hirsutism, obesity, inflammation, insulin resistance and type 2 diabetes (Lizneva et al. 2016, Norman et al. 2007, Dimitriadis et al. 2016). PCOS is also characterized by the accumulation of numerous cysts (fluid-filled sacs) on the ovaries. First line therapy includes treatment with oral contraceptive therapy to control the hyperandrogenemia (Legro et al. 2013), with clomiphene used as first line treatment for fertility treatment (Vause et al. 2010). Lifestyle modifications and/or weight loss have been found to be effective in improving fertility and may be associated with improved insulin sensitivity (Orio et al. 2016).

PCOS is also the most common obesity-related endocrine syndrome in women. In the United States, some studies report that the prevalence of overweight and obesity in women with PCOS is as high as 80%. Outside the U.S., the prevalence of obesity in affected women is lower, although it has increased over time, with studies reporting rates of about 20%. The interaction of both environmental factors, such as lifestyle, and diverse genetic factors are thought to contribute to development of obesity in PCOS.

Due, at least in part, to the complex phenotype of PCOS, appropriate therapies are still needed.

### SUMMARY

In some aspects the present disclosure provides methods for treating PCOS in a mammal in need thereof, the method comprising administering to the mammal a therapeutically effective amount of a tetrahydro-iso-alpha acid (THIAA) derivative and/or salt thereof. In certain of these embodiments, the methods comprise preventing or delaying the development of one or more symptoms associated with PCOS, and/or reducing or eliminating one or more symptoms associated with PCOS.

In some embodiments, the PCOS is non-insulin resistant PCOS.

In some embodiments, the mammal is administered a chalcone with the THIAA derivative and/or salt thereof. In other embodiments, the mammal is administered metformin with the THIAA derivative and/or salt thereof.

In some embodiments, the symptoms associated with PCOS being prevented, delayed, reduced, and/or ended include one or more of increased insulin levels and/or sensitivity, ovarian inflammation, increased blood glucose, increased serum lipid levels, increased testosterone levels, impaired fertility, amenorrhea, oligomenorrhea, and anovulation. In some of these embodiments, the serum lipid levels are cholesterol and/or triglycerides. In some of these embodiments, the impaired fertility is detected by measuring at least one marker of fertility, for example, cyclicity and/or ovulation.

In some embodiments, the THIAA derivative and/or salt thereof has a direct effect on ovarian function.

In some embodiments, the mammal exhibits a decrease in liver fat content.

In some embodiments, the THIAA derivative and/or salt thereof comprises KDT500, KDT501, or a combination thereof. In some embodiments, the KDT501 is enantiomerically pure (+)KDT501.

In some embodiments, the THIAA derivative and/or salt thereof is administered to the mammal once a day. In other embodiments, the THIAA derivative and/or salt thereof derivative is administered to the mammal two or more times per day.

In some embodiments, the THIAA derivative and/or salt thereof is a synthetic derivative of a tetrahydroisohumulone scaffold, THIAA derivative, and/or salt thereof.

### BRIEF DESCRIPTION OF DRAWINGS

FIG. 1 demonstrates that KDT501 attenuated weight gain in female mice on a high fat diet as compared to control mice treated with pioglitazone (PIO) or vehicle (VEH). Female mice were fed 60% HFD (60% calories from fat) for 80 days. Mice were treated with VEH, KDT501 (KDT; 100 mpk) or PIO (30 mpk) and weight assessed for 8 weeks. Data are presented as Δ weight (weight gain) from week 0 over the eight week period

FIG. 2A demonstrates that morning levels of LH and FSH were not significantly altered by treatment with PIO or KDT501 when compared to VEH under the tested conditions.

FIG. 2B demonstrates no significant difference was observed in the ratio of LH/FSH between KDT501- and PIO-treated mice as compared to VEH under the tested conditions.

FIG. 3A shows representative images of sectioned and H&E stained ovaries from VEH-, KDT501-, and PIO-treated mice.

FIG. 3B show average number of corpora lutea in ovaries from obese mice treated with VEH, KDT501, or PIO. Significantly more corpora lutea (CL) were observed in ovaries from mice treated with PIO and KDT501 than in VEH mice. Average number of CL counted from five consecutive sections in each female.

FIG. 4A shows percentage of mice that completed 1, 2 or 3 complete estrous cycles during the 15 day period. Estrous cyclicity was assessed by examination of vaginal cytology. Estrous cycle staging occurred over a 15 day period. Mice were scored as having either one, two or three complete estrous cycles over the 15 days. Roughly an even percentage of VEH, PIO and KDT501 treated mice had one complete cycle in 16 days; however 60% of KDT501 treated mice had at least two complete cycles while only 25-28% of VEH and PIO treated mice had two complete cycles. No VEH treated mice had 3 complete cycles, while 28 and 20%, respectively; of PIO and KDT501 treated mice had 3 complete cycles.

FIG. 4B shows the average number of days +/−SEM of the total number of cycles each mouse completed during the 16 days of analysis. KDT501 mice had a significant increase in number of cycles/mouse. Data are displayed as mean +/−SEM.

FIG. 5A shows that treatment with KDT501 or PIO resulted in improved glucose tolerance relative to VEH treated mice. Glucose metabolism was assessed by IP-GTT. IP-GTT performed using one unit (0.01 cc)/g BW of a 20% dextrose solution. Glucose levels measured in blood obtained from tail vein using One Touch Glucometer at different time points.

FIG. 5B demonstrates that while there was a significant improvement in glucose tolerance in KDT501 treated mice, glucose tolerance was not significantly improved by treatment with PIO. AUC plotted for each group.

FIG. 6A depicts assessment of whole body insulin sensitivity using insulin tolerance test (ITT). Both KDT501 and PIO treated mice had lower fasting glucose levels (7 hrs fasting) than VEH controls. Glucose levels are plotted at various time points following an IP injection of insulin (0.3 Units/kg body weight).

FIG. 6B show the area under the curve (AUC) for each mouse analyzed in FIG. 6B. Results show that both PIO- and KDT501-treated obese female mice exhibit improved insulin sensitivity relative to VEH controls.

FIG. 7A is a representative image of H&E staining demonstrating lipid accumulation in livers of KDT501-treated mice.

FIG. 7B is a representative image of H&E staining demonstrating lipid accumulation in livers of VEH-treated mice.

FIG. 7C is a representative image of H&E staining demonstrating lipid accumulation in livers of PIO-treated mice.

FIG. 7D is a representative image of H&E staining demonstrating lipid accumulation in livers of PIO-treated mice. Due to the increased variation of histological appearance of the livers from PIO treated female mice, an additional H&E staining of liver section from PIO treated female mouse is displayed

FIG. 7E quantifies the lipid accumulation in VEH-, KDT501-, and PIO-treated obese mice on a high fat diet. ImageJ software was used to quantitate fat content in liver sections. Arbitrary unit of fat as assessed by area whiteness is plotted. There was a significant reduction in fat in livers from PIO and KDT501 treated females relative to VEH treated females. Additionally, there was significantly less fat in the livers of KDT501 treated females than PIO treated females.

FIG. 8 shows results from analysis of serum hormone levels. Blood was obtained in the morning from fasted females and measure by multiplex ligand Luminex assay. No significant differences in serum insulin (FIG. 8A), leptin (FIG. 8B) or IL-6 (FIG. 8C) levels between VEH, KDT501 or PIO treated obese female mice.

### DETAILED DESCRIPTION

The following description is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

As disclosed herein, a novel use of a THIAA derivative and/or salt thereof, for example, KDT501, for treating PCOS has been discovered. Provided herein in certain embodiments are the use of THIAA derivatives and salts and crystals thereof, including crystals of the salts for the treatment of PCOS. Definitions

The term “about” when used before a numerical designation, e.g., temperature, time, amount, concentration, and such other, including a range, indicates approximations which may vary by (+) or (−) 20%, 10%, 5% or 1%.

The term “and/or” as used herein refers to and encompasses any and all possible combinations of one or more of the associated listed items, as well as the lack of combinations when interpreted in the alternative (“or”).

“Comprising” or “comprises” is intended to mean that the compositions of, for example THIAAs, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The terms “administering,” “administer” and the like refer to introducing an agent (e.g., a THIAA) into a mammal. Typically, a therapeutically effective amount is administered, which amount can be determined by the treating physician or the like. Any route of administration, such as topical, subcutaneous, peritoneal, intravenous, intraarterial, inhalation, vaginal, rectal, nasal, oral, buccal, or instillation into body compartments can be used. The terms and phrases “administering” and “administration of,” when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer an agent (e.g., a THIAA) and/or provides a patient with a prescription for a drug is administering the agent to the patient. “Periodic administration” or “periodically administering” refers to multiple treatments that occur on a daily, weekly, or a monthly basis. Periodic administration may also refer to administration of an agent one, two, three or more time(s) per day.

The term “salt” as used herein may refer to any pharmaceutically acceptable salt, including for example inorganic base salts such as potassium, aluminum, calcium, copper, guanidinium, iron, lithium, magnesium, sodium, and zinc salts and organic base salts such as cinchonidine, cinchonine, and diethanolamine salts. Additional examples of pharmaceutically acceptable salts and preparations in accordance with the present invention can be found in, for example, Berge J Pharm Sci 66: 1 (1977).

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

The terms “treat,” “treating,” or “treatment” as used herein with regards to a condition (e.g., PCOS) refers to preventing the condition, eliminating the condition, slowing the onset or rate of development of the condition, reducing the risk of developing the condition, preventing or delaying the development of one or more symptoms associated with the condition, reducing or eliminating one or more symptoms associated with the condition, or some combination thereof

A “therapeutically effective amount” of a THIAA derivative, salt thereof, and/or pharmaceutical composition as used herein is an amount of a composition that produces a desired therapeutic effect in a subject. The precise therapeutically effective amount is an amount of the compound or composition that will yield the most effective results in terms of therapeutic efficacy in a given subject. This amount will vary depending upon a variety of factors, including but not limited to the characteristics of the therapeutic compound (including, e.g., activity, pharmacokinetics, pharmacodynamics, and bioavailability), the physiological condition of the subject (including, e.g., age, sex, disease type and stage, general physical condition, responsiveness to a given dosage, and type of medication), the nature of the pharmaceutically acceptable carrier or carriers in the composition, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005, the entire disclosure of which is incorporated by reference herein.

Provided herein in certain embodiments are compositions comprising one or more of the THIAA derivatives provided herein. In certain of these embodiments, the compositions are substantially enantiomerically pure. The term “substantially enantiomerically pure” as used herein refers to a composition in which 90% or more of a particular compound in the composition is in a first enantiomeric form, while 10% or less is in a second enantiomeric form. In certain embodiments, the “first enantiomeric form” of a compound includes salts and crystals of that enantiomeric form. In certain embodiments, a substantially enantiomerically composition may contain 91%, 92%, 93%, 94%, 95%, 96%, 97%, 98%, 99%, 99.5%, 99.9%, 99.95%, or 99.99% or greater of a first enantiomeric form of a compound.

### Hops Derivatives

Hops (Humulus lupulus L.) is a plant that has been used for medicinal purposes for centuries and is currently used in the brewing industry. Hops contains both alpha acids (humulones) and beta acids (lupulones). Alpha acids/humulones have the general structure:

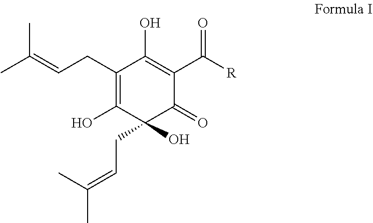

The three primary types of alpha acids are humulone (R═CH₂CH(CH₃)₂), cohumulone (R═CH(CH₃)₂), and adhumulone (R═CH(CH₃)CH₂CH₃). There are also two less common alpha acids in hops, prehumulone and posthumulone. Alpha acids can be converted to cis or trans iso-alpha acids/isohumulones by heat-induced isomerization of alpha acids, and these iso-alpha acids can in turn converted to cis or trans reduced iso-alpha acids by hydrogenation. The three primary types of reduced iso-alpha acids are dihydro- (also known as rho-), tetrahydro-, and hexahydro-iso-alpha acids (RIAA, THIAA, and HIAA, respectively).

Several compounds derived from hops have been found to possess anti-inflammatory activity (Hall 2008; Desai 2009; Tripp 2009; Konda 2010). THIAA extracts have been shown to inhibit inflammation (Desai 2009), reduce symptoms of arthritis in a mouse model of collagen-induced arthritis (Konda 2010), and improve glucose homeostasis in a high fat diet-induced metabolic endotoxemia model (Everard 2012). In each of these cases, the THIAA compounds shared a substituted 1,3-cyclopentadione motif.

The THIAA cis 3,4-dihydroxy-2-(3-methylbutanoyl)-5-(3-methylbutyl)-4-(4-methylpentanoyl)cyclopent-2-en-1-one (“KDT500,” also known as cis tetrahydro isohumulone) has two enantiomers: (+)-(4S,5R)-3,4-dihydroxy-2-(3-methylbutanoyl)-5 -(3-methylbutyl)-4-(4-methylpentanoyl)cyclopent-2-en-1-one (“(+)-KDT500”) and (−)-(4R,5S)-3,4-dihydroxy-2-(3-methylbutanoyl)-5-(3-methylbutyl)-4-(4-methylpentanoyl)cyclopent-2-en-1-one (“(−)-KDT500”). The structures of (+)-KDT500 and (−)-KDT500, non-limiting examples of derivatives of a THIAA derivative and/or salt thereof, are set forth in Formulas II and III, respectively.

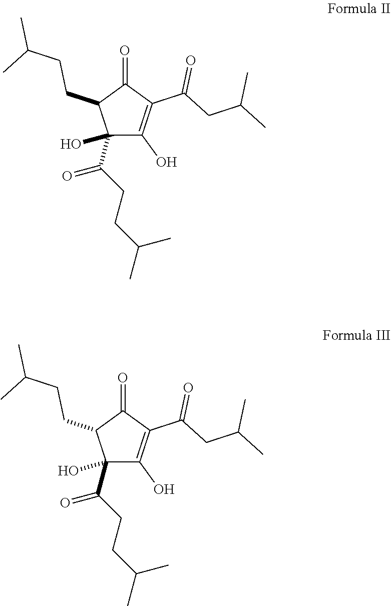

U.S. Pat. Nos. 8,410,178, 8,410,179, and 8,829,056, the disclosures of which are hereby incorporated by reference herein in their entirety, describe the purification and characterization of KDT500 and its potassium salt KDT501, a THIAA derivative and/or salt thereof An enriched THIAA extract containing predominantly the cis diastereomers was obtained during hops processing and purified using countercurrent chromatography (CCC), and the isolated (+)-KDT500 was converted to (+)-KDT501 by reacting with 1 equivalent of potassium salt (e.g., KOH).

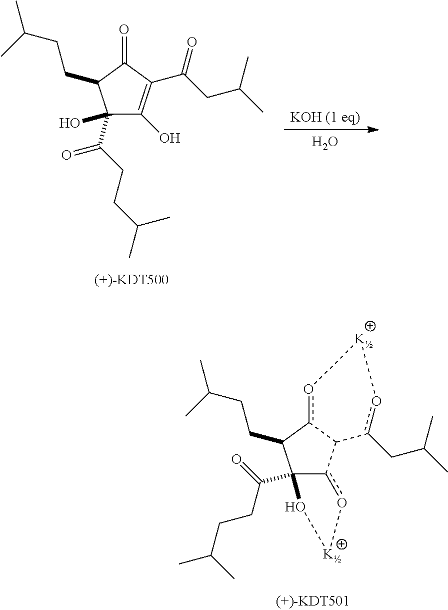

U.S. Pat. No. 9,340,479, the disclosure of which is hereby incorporated by reference herein in its entirety, describes additional methods of making certain KDT500 derivatives, including KDT501, and enantiomerically pure compositions thereof.

(+)-KDT501 has been shown previously to exhibit both anti-inflammatory and anti-diabetic effects.

In some embodiments of the methods, compositions, and kits provided herein, the THIAA is KDT500, KDT501, or a combination thereof. In some embodiments, the KDT501 is an enantiomerically pure compositions of (+)KDT501.

TABLE 1
KDT500 and KDT501

| Compound | | | |
|---|---|---|---|
| name | MW | Structure | Chemical name |
| (+)-KDT500 | 366 | 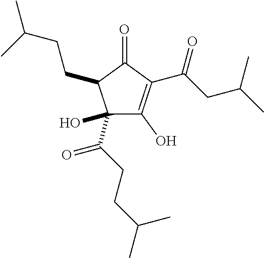 [SMILES: CC(C)CCC(=O)[C@@]1(O)C(O)=C(C(=O)CC(C)C)C(=O)[C@@H]1CCC(C)C] | (+)-(4S,5R)-3,4-dihydroxy-2-(3- methylbutanoyl)-5-(3-methylbutyl)-4- (4-methylpentanoyl)cyclopent-2-en-1- one |
| (+)-KDT501 | 404 | 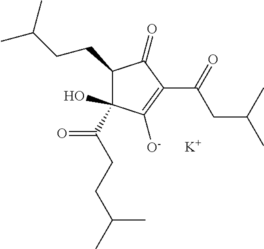 [SMILES: CC(C)CCC(=O)[C@@]1(O)C([O-])=C(C(=O)CC(C)C)C(=O)[C@@H]1CCC(C)C ; [K+]] | Potassium salt of (+)-KDT500 |

### Compositions

In certain embodiments the present disclosure provides compositions, including pharmaceutical compositions, comprising one or more of the THIAA derivatives provided herein and one or more pharmaceutically acceptable carriers. In certain embodiments, the compositions are substantially enantiomerically pure. In certain embodiments, a substantially enantiomerically composition may contain 91%, 92%, 93%, 94%, 95%, 96%, 97%, 98%, 99%, 99.5%, 99.9%, 99.95%, or 99.99% or greater of a first enantiomeric form of a compound (i.e., the THIAA derivative and/or salt thereof).

The concentration of substantially enantiomerically-pure THIAA derivative and/or salt thereof (i.e., KDT501) in the compositions provided herein may vary. Concentrations may be selected based on fluid volumes, viscosities, body weight, and the like in accordance with the particular mode of administration selected and the biological system's needs. In certain embodiments, the concentration of substantially enantiomerically-pure THIAA derivative and/or salt thereof in a composition provided herein may be from about 0.0001% to 100%, from about 0.001% to about 50%, from about 0.01% to about 30%, from about 0.1% to about 20%, or from about 1% to about 10% wt/vol.

In certain embodiments, pharmaceutical compositions of the THIAA derivatives provided herein comprise a pharmaceutically acceptable carrier. A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid or solid filler, diluent, excipient, solvent, encapsulating material, stabilizing agent, or some combination thereof Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Examples of pharmaceutically acceptable carriers for use in the compositions provided herein include, but are not limited to, (1) sugars, such as lactose, glucose, sucrose, or mannitol; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) disintegrating agents such as agar or calcium carbonate; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; (21) paraffin; (22) lubricants, such as talc, calcium stearate, magnesium stearate, solid polyethylene glycol, or sodium lauryl sulfate; (23) coloring agents; (24) glidants such as colloidal silicon dioxide, talc, and starch or tri-basic calcium phosphate; and (24) other non-toxic compatible substances employed in pharmaceutical compositions such as acetone. In one embodiment, the pharmaceutically acceptable carrier used herein is an aqueous carrier, e.g., buffered saline and the like. In other embodiments, the pharmaceutically acceptable carrier is a polar solvent, e.g., acetone and alcohol.

Pharmaceutical compositions as provided herein may further comprise one or more pharmaceutically acceptable auxiliary substances as required to approximate physiological conditions. For example, compositions may comprise one or more pH adjusting agents, buffering agents, or toxicity adjusting agents, including for example sodium acetate, sodium chloride, potassium chloride, calcium chloride, sodium lactate, and the like.

Pharmaceutical compositions as provided herein may be formulated into a suitable dosage form, including for example capsules, cachets, pills, tablets, lozenges (using a flavored basis, usually sucrose and acacia or tragacanth), powders, granules, as a solution or a suspension in an aqueous or non-aqueous liquid, as an oil-in-water or water-in-oil liquid emulsion, as an elixir or syrup, or as pastilles (using an inert base, such as gelatin and glycerin, or sucrose and acacia) and/or as mouth washes and the like, each containing a predetermined amount of a THIAA derivative as an active ingredient. In certain embodiments, the compositions may be formulated as a time release delivery vehicle, such as for example a time release capsule. A “time release vehicle” as used herein refers to any delivery vehicle that releases active agent over a period of time rather than immediately upon administration. In other embodiments, the compositions may be formulated as an immediate release delivery vehicle.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a substantially enantiomerically pure mixture of the powdered THIAA derivative or further moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of a THIAA derivative therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain pacifying agents and may be of a composition that they release the THIAA derivative(s) only, or preferentially, in a certain portion of the gastrointestinal tract, optionally, in a delayed manner. Examples of embedding compositions which can be used include polymeric substances and waxes. The THIAA derivative can also be in micro-encapsulated form, if appropriate, with one or more of the above-described excipients.

The composition can be included in an implantable device. Suitable implantable devices contemplated by this invention include intravascular stents (e.g., self-expandable stents, balloon-expandable stents, and stent-grafts), scaffolds, grafts, and the like. Such implantable devices can be coated on at least one surface, or impregnated, with a composition of the present disclosure.

The compositions can be incorporated into any drug delivery system known to one of skill in the art, including nanosized systems. Non-limiting examples of nanosized systems include nanoparticles, micelles, liposomes and drug conjugates, microspheres, implants, and injectable depots.

The concentration of THIAA derivatives in the compositions provided herein may vary. Concentrations may be selected based on fluid volumes, viscosities, body weight, and the like in accordance with the particular mode of administration selected and the biological system's needs. In certain embodiments, the concentration of a THIAA derivative in a composition provided herein may be from about 0.0001% to 100%, from about 0.001% to about 50%, from about 0.01% to about 30%, from about 0.1% to about 20%, or from about 1% to about 10% wt/vol.

In certain embodiments, the compositions comprise a single enantiomer of a THIAA derivative. In other embodiments, the compositions comprise a mixture of enantiomeric forms of THIAA derivatives.

### Methods of Treatment

The present disclosure provides methods of treating polycystic ovary syndrome (PCOS) and/or symptoms associated with PCOS in a subject (e.g., a mammal) in need thereof, the methods comprising administering to the mammal an amount of a THIAA derivative and/or salt thereof

In certain embodiments of the methods provided herein, the subject is a mammal, and in certain of these embodiments the mammal is a human. A “subject in need thereof” refers to a subject diagnosed with PCOS or a condition associated with PCOS, a subject who exhibits or has exhibited one or more symptoms of PCOS or a condition associated with PCOS, or a subject who has been deemed at risk of developing PCOS or a condition associated with PCOS. Non-limiting examples of symptoms associated with PCOS include increased insulin levels and/or sensitivity, increased blood glucose levels, increased serum lipid levels (e.g., cholesterol and/or triglycerides), increased testosterone levels, impaired fertility, amenorrhea, oligomenorrhea, anoluvation, increased liver fat content, or any combination thereof

In some embodiments the PCOS is non-insulin resistant PCOS. Insulin-resistant PCOS is sometimes referred to as Type I PCOS. Patients with Type I PCOS often develop classic symptoms of PCOS including, for example, weight gain, ovulatory interruptions, facial hair, hair loss, acne, increased testosterone, and a greater potential for developing diabetes. Patients also may experience insulin and leptin resistance. Non-insulin resistant PCOS, or Type II PCOS, occurs in patients who meet diagnostic criteria for PCOS, but do not present with insulin resistance. While Type I and Type II PCOS are the most common, non-traditional PCOS I, non-traditional PCOS II, and idiopathic hirsutism are other known variations. In some embodiments, one or more variations of PCOS are expressly excluded from the present disclosure.

In certain embodiments, a compound or composition as provided herein may be administered one or more times a day. In other embodiments, the compound or composition may be delivered less than once a day. For example, the compound or composition may be administered once a week, once a month, or once every several months. Administration of a compound or composition provided herein may be carried out over a specific treatment period determined in advance, or it may be carried out indefinitely or until a specific therapeutic benchmark is reached. In certain embodiments, dosing frequency may change over the course of treatment. For example, a subject may receive less frequent administrations over the course of treatment as certain therapeutic benchmarks are met. In one embodiment, the THIAA derivative and/or salt thereof is administered to the subject daily for a period of at least about 2 weeks, at least about 4 weeks, or at least about 8 weeks.

In some embodiments, THIAA derivative and/or salt thereof is the sole agent administered for the treatment of PCOS. In some embodiments, the THIAA derivative and/or salt thereof is administered in combination with a second agent. Non-limiting examples of suitable second agents include, medications to lower blood sugar, a hormone therapy (e.g., birth control pills, patches, or vaginal rings), natural alternatives (e.g., D-Chiro-Inositol), iodine, vitamin D, magnesium, zinc, natural progesterone, herbal formulations to reduce testosterone, androgen-lowering spironolactone, metformin, clomiphene, or any combination thereof. In other embodiments, one or more second agents are expressly excluded, for example, one or more chalcones (e.g., methylhydroxy chalcone polymer (MHCP)) or derivatives thereof and/or metformin. The THIAA derivative and/or salt thereof and second agent can be administered sequentially or in combination.

In certain embodiments, upon treatment in accordance with the present disclosure, for example, over a period of time the subject or subject group exhibits one or more of the following outcomes:

(a) an improvement in a symptom or plurality of symptoms of PCOS as reported by the subject of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(b) a reduction in number of days in the preceding week in which the subject reported that their sleep was disturbed due to sleep apnea of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(c) an increase in number of corpora luteum as detected by, for example, ultrasonography or magnetic resonance imaging (MRI), as compared to baseline, a reference, or placebo control of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(d) improved menstrual cyclicity, as measured by number of cycles and/or regularity of cycles compared to baseline, a reference, or placebo control;

(e) an improvement in glucose metabolism as detected by, for example, blood glucose levels during glucose tolerance test (GTT) as compared to baseline, a reference, or placebo control;

(f) an increase in weight loss, particularly in the abdominal region, as compared to baseline, a reference, or placebo control of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(g) improved insulin tolerance detected, for example, by lower fasting glucose levels, compared to baseline, a reference, or placebo control of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(h) a decrease in fat content in liver compared to baseline, a reference, or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(i) a decrease in serum lipid levels (e.g., cholesterol and/or triglycerides) compared to baseline, a reference, or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(j) a decrease in expression of at least one marker of inflammation (e.g., TNF-α, adiponectin) compared to baseline, a reference, or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(k) an increase in enhancement of brown fat, as detected, for example, by a fat biopsy, as compared to baseline, a reference, or placebo control of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(k) an increase in the level of high molecular weight (HMW) adiponectin, as detected, for example, in serum, as compared to baseline, a reference, or placebo control of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%; and/or

(l) measurable weight loss, particularly in abdominal region, as compared to baseline, a reference, or placebo control within at least 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, 2 years, 3 years, 4 years, or 5 years.

In certain embodiments, upon treatment in accordance with the present disclosure, for example, over a period of time the subject or subject group exhibits no or substantially no change in pituitary function compared to baseline, a reference, or placebo control. Pituitary function can be assessment by any method known to one of skill in the art including, for example, measurement of pituitary gonadotrophin levels (e.g., luteinizing hormone (LH), follicle stimulating hormone (FSH), and ratios thereof).

In certain embodiments, upon treatment in accordance with the present disclosure, for example, over a period of time the subject or subject group exhibits no or substantially no change in serum hormone levels compared to baseline, a reference, or placebo control. Serum hormone levels (e.g., serum insulin, leptin, interleukin-6 (IL-6)) can be assessed by any method known to one of skill in the art including, for example, bead-based multiplex assays.

As used herein the term “baseline” refers to a level detected in a patient or measured in a biological sample collected from the patient at a point prior to the current treatment. The term “reference” refers to a level detected in a patient or population of patients that are known to suffer from PCOS or known to not suffer from PCOS.

In some embodiments, the THIAA derivative and/or salt thereof has a direct effect on ovarian function. In some embodiments, it is contemplated that the direct effect is responsible or partially responsible for one or more of the above outcomes, for example, improved cyclicity and/or increased number of corpora lutea. In some embodiments, it is contemplated that the direct effect is achieved through the bitter taste receptors.

### Kits

In certain embodiments, kits are provided that comprise one or more of the THIAA derivatives and/or salts thereof , pharmaceutical formulations, or substantially enantiomerically pure compositions provided herein. In certain embodiments, the kit provides instructions for usage, such as dosage or administration instructions. In certain embodiments, the kits may be used to treat a condition associated with PCOS.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: KDT501 Improves Metabolic and Reproductive Function in Diet-Induced Obesity (DIO) Female Mice

### KDT501 Attenuated Weight Gain on HFD in Female Mice

To generate DIO mice, 7 week old female mice on a mixed strain background (129SvJ, C57B16, CD1) were placed and maintained on a high fat diet (HFD) for 8 weeks prior to study and throughout the course of study. The high fat diet consisted of 60% Kcal from fat with an energy density of 5.24 kcal/gm (D12492, Research Diets, Inc., New Brunswick, N.J., USA).

Wood chip bedding was maintained in these cages so that mice would not supplement their diet by eating the standard corncob bedding. Mice were weighed every week. The regular chow diet (Tekland Global 18% protein diet) was 24% Kcal from protein, 58% Kcal from carbohydrate, and 18% Kcal from fat with an energy density of 3.1 kcal/g (Harlan Laboratories, Indianapolis, Ind., USA). All procedures were performed with approval by the Johns Hopkins Animal Care and Use Committee under standard light and dark cycles.

At 80 days after introduction to high fat diet, the test compounds were administered orally to the mice once a day. Mice were randomly allocated on the basis of body weight to three groups (each group between 10-14 mice). KDT501 (100 mg/kg), Pioglitazone (PIO; 30 mg/kg) and Vehicle (VEH; 0.5% methylcellulose and 0.2% Tween 80 (w/v)).

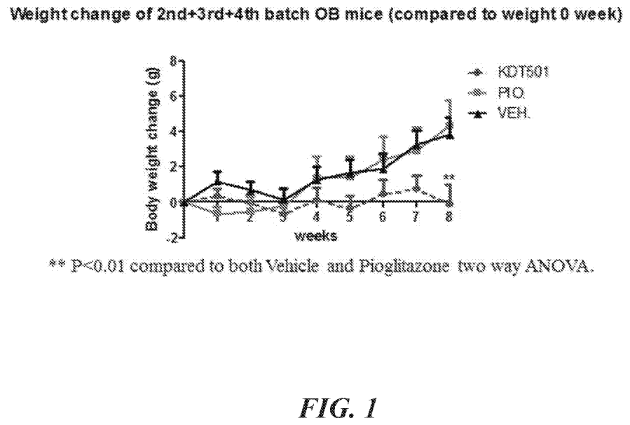

Female mice fed a high fat diet (60% calories from fat) gained approximately 4 grams of weight during the 8 week study period (FIG. 1). There was no difference in weight gain between mice treated with PIO or vehicle VEH. KDT501 treated mice exhibited no weight gain over the 8 week study period, and their change in weight was significantly attenuated when compared to PIO or VEH treated mice. All data were analyzed with using Prism Software 5 GraphPad Software (La Jolla, CA) and are expressed as means ±SEM. Significance was determined via unpaired two- tailed Student t-test, one- or two-way ANOVA with the appropriate post-hoc tests. P>0.05 was defined as statistically significant

### KDT501 Did Not Change Pituitary Gonadotropin Levels

To measure serum levels of LH, FSH, insulin, and leptin, serum samples were collected from mice via mandibular bleed. Mice were fasted overnight, and insulin or saline was injected IP according to the body weight of the mice (0.3 units/kg). Samples were obtained between 9:00 and 10:00 am on diestrus for baseline LH and FSH so as to avoid cycle dependent LH surges that occur in the late afternoon on the evening of proestrus. Rodent morning LH levels are not thought to vary in a cycle dependent manner (Helena et al. 2006). For fasting measurements of insulin, samples were also obtained between 9:00 and 10:00 am. Tissues were collected 10 minutes after insulin injection and snap-frozen in liquid nitrogen. Protein was obtained and measured as described previously (Brothers et al. 2010). To quantify the levels of phosphorylated AKT (pAKT), and AKT for different tissues of each individual animal after insulin or saline injection, Bio-Plex Phosphoprotein Detection Multiplex assays were used (Bio-Rad Laboratories, Hercules, Calif.). Protein (10 mg) from each tissue was loaded into a 96-well microplate. Assays were conducted using the Luminex 200 system. Values were assessed as mean fluorescent intensity of phospho-specific signal. Serum was analyzed on a Luminex 20015 platform using the Milliplex Map mouse pituitary panel (LH/FSH) and a Milliplex mouse metabolic panel (insulin/leptin; Millipore, Billerica, Mass., USA). The assay detection limit for LH was 0.0019 ng/mL; for FSH, 0.0095 ng/mL; for insulin, 13.0 pg/mL; and for leptin, 4.2 pg/mL. Serum Testosterone was measured by radioimmunoassay at the University of Virginia Ligand Assay Core.

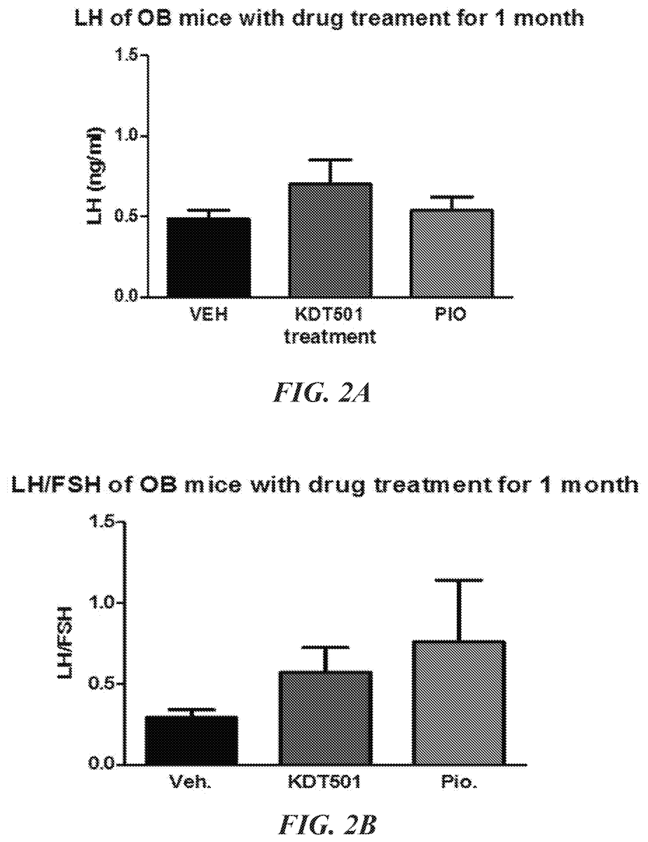

Results showed that morning levels of LH and FSH were not significantly altered by treatment with PIO or KDT501 when compared to VEH (FIG. 2A). Additionally, there was no significant difference in LH and FSH levels between KDT501 and PIO treated mice. Since the ratio of LH/FSH can impact ovarian steroidogenesis, this ratio was calculated and no significant difference between groups was observed (FIG. 2B). It is contemplated that an absence or non-significant difference in hormonal changes improves the clinical efficacy of KDT501 in comparison to other compounds. In particular, hormonal changes in women correspond with delayed ovulation, decreased endometrial thickening, impeded follicular maturation, and longer menstrual cycles. A lack of effect on LH/FSH implicates the KDT501 activity as not primary to the central nervous system and thus avoids associated safety risks.

### KDT501 and PIO Increase Number of Corpora Lutea in DIO Female Mice

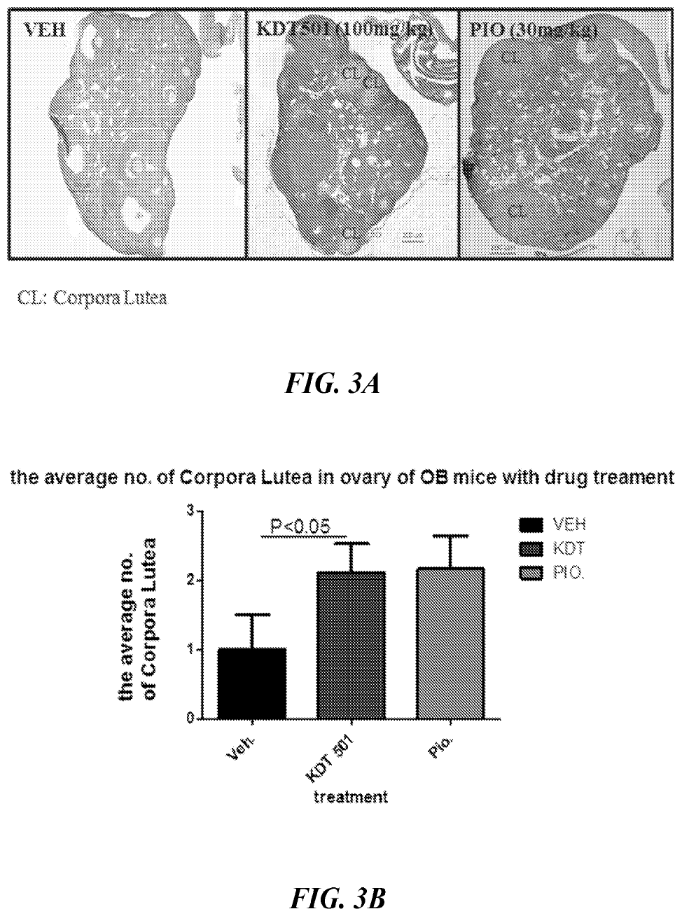

Sectioned and stained ovaries (FIG. 3A) were evaluated for numbers of corpora lutea. Ovaries were collected and placed immediately in 10% formalin. Ovaries were sectioned to 5 μm thickness and collected every 10th section for total 10 and stained with H&E by the Johns Hopkins Molecular and Comparative Pathobiology Phenotyping Core. Ovary tissue from each group of mice was paraffin sectioned by the Johns Hopkins University Pathology Core Facility. 5 μm sections were stained with rat anti-mouse F4/80 (1:100; Cedarlane, Burlington, Ontario, Calif.) biomarkers for macrophage infiltration (Kiefer et al. 2010). Secondary antibody Alexa Fluor 594 goat anti-rat IgG (H+L) (1:400) was obtained from Invitrogen by Thermo Fisher Scientific (Rockford, Ill., USA). The total number of corpora lutea counted per mouse were compared across groups. Corpora lutea develop from follicles that have ovulated and therefore represent the recent ovulatory history of the mouse. Few CL were observed in VEH treated mice, as expected based on our previous studies (Brothers et al. 2010, Wu et al. 2014). There were significantly more CL present in ovaries from mice treated with KDT501 than in VEH mice (FIG. 3B).

### Estrous Cyclicity Improved in KDT501 and PIO Treated DIO Female Mice

Diet-induced obesity has been reported to impair estrous cyclicity in female mice (Brothers et al., Wu et al.). In these studies, DIO females were treated with KDT501, PIO or VEH beginning 8 weeks after change to high fat diet. Mice were assessed for estrous cycle stage for 15 consecutive days beginning 10 days after drug/VEH treatment. Vaginal cytology was assessed daily between 9 and 10 am for 15 consecutive days beginning the 10th days after drug treatment. Vaginal cells were collected via saline lavage and then fixed with 100% ethanol and stained with the DIFF Quick Stain Kit (IMEB Inc., San Marcos, Calif., USA). Stages were assessed based on vaginal cytology (Nelson et al. 1982): predominant cornified epithelium indicated the estrus stage, predominant nucleated cells indicated the proestrus stage, both cornified and leukocytes indicated the metestrus stage, and predominant leukocytes indicated the diestrus stage. The percentage of mice at each different cycle stage in each group was calculated.

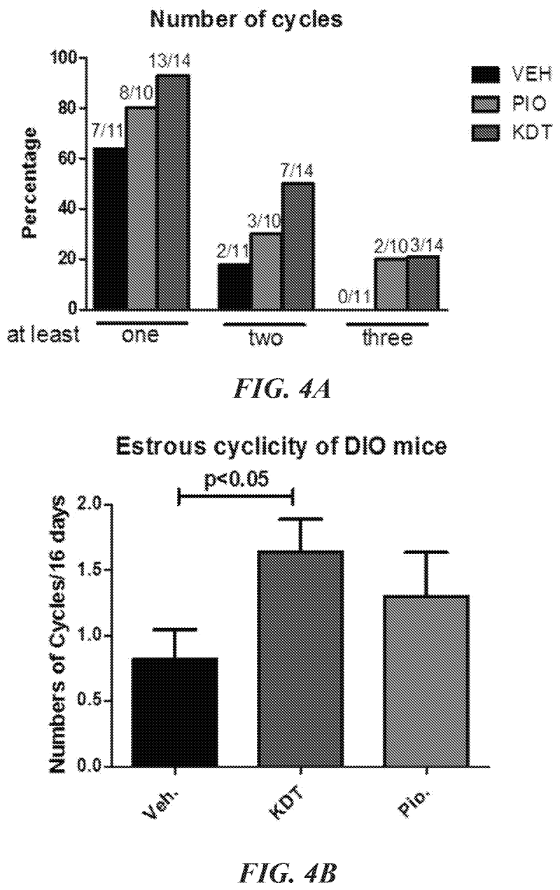

FIG. 4A plots the percent of mice that completed 1, 2 or 3 complete estrous cycles during the 15 day period. 37% of VEH treated mice did not complete even 1 complete estrous cycle. Treatment with PIO resulted in 20% of mice being acyclic, and only 7.1% of KDT501 treated DIO females were acyclic. No VEH treated DIO female completed 3 complete estrous cycles while 20% ( 2/10) of KDT501 and 21% ( 3/14) of PIO treated mice completed 3 complete estrous cycles. These data suggest a modest improvement in estrous cyclicity in PIO treated mice compared to VEH treated controls, while a more dramatic improvement in estrous cyclicity, relative to VEH control, results from treatment with KDT501. In FIG. 4B the number of cycles each mouse completed during the 15 days of analysis was plotted. The KDT501 treated DIO females had significantly more complete estrous cycles than VEH treated mice. PIO treated DIO female mice also had more complete cycles than VEH treated mice, but the difference was not significant.

### Glucose and Insulin Tolerance in DIO Female Mice

To begin to assess the effects of drug treatment on glucose metabolism in female DIO mice, IP-glucose tolerance test (IP-GTT) was performed. One month after drug treatment, mice were tested for glucose tolerance by IP-GTT. The mice were fasted overnight for 16 hours, blood was drawn through the tail vein and baseline blood glucose was measured using a OneTouch Ultra glucometer. 20% dextrose was injected IP (2 g/kg body weight). Sample blood glucose was measured at 15, 30, 60, 90 and 120 minutes. One week after IP-GTT, the mice were tested for whole body insulin sensitivity by insulin tolerance test (ITT). Mice were fasted for seven hours, blood was drawn through the tail vein and baseline blood glucose was measured as above. 0.3 units/kg insulin was injected IP. Blood glucose of each mouse were measured at 15, 30, 60, 90 and 120 minutes. Area under the curve (AUC) was calculated for both the IP-GTT and the ITT.

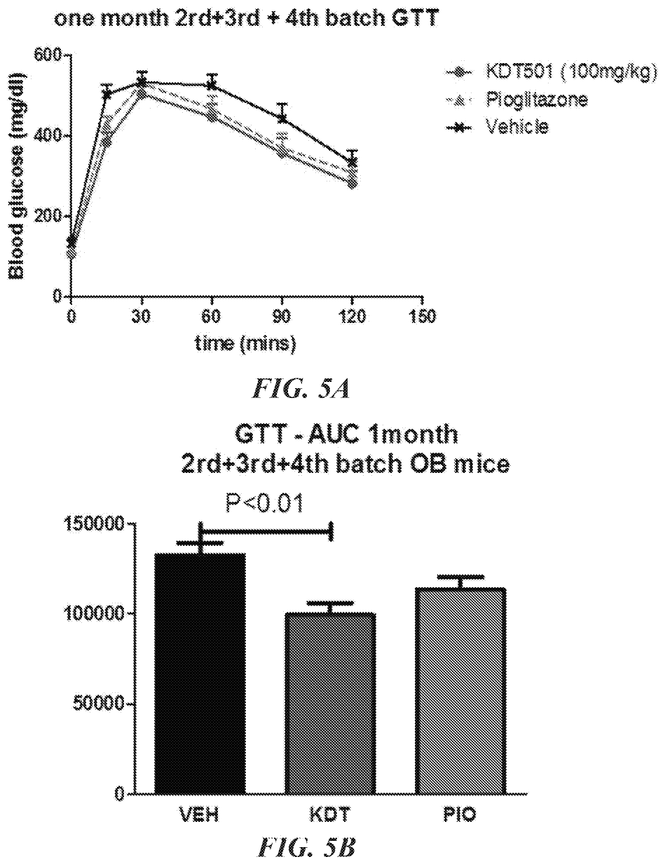

Glucose tolerance in response to injection with glucose is impaired in DIO female mice compared to chow fed females (Wu et al. 2012). Treatment with KDT501 or PIO resulted in improved glucose tolerance relative to VEH treated mice (FIG. 5A). When area under the curve data was calculated, there was a significant improvement in glucose tolerance in KDT501 treated mice. While improved, glucose tolerance was not significantly improved by treatment with PIO (FIG. 5B).

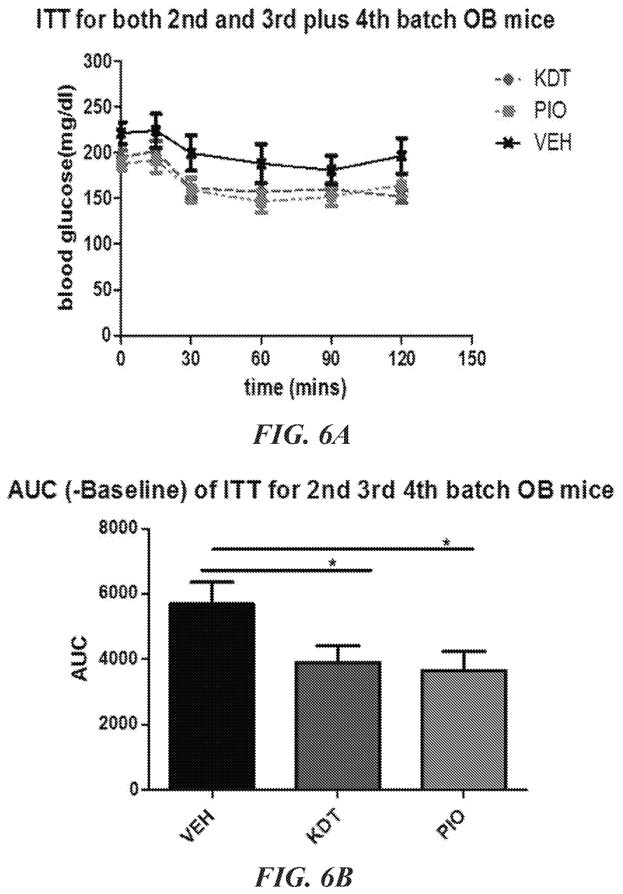

Whole body insulin sensitivity was assessed by an insulin tolerance test (ITT). Glucose levels were measured following an injection of insulin (FIG. 6A). DIO females exhibit impaired insulin sensitivity when compared to chow fed females (Wu et al. 2012). Area under the curve (AUC) was calculated for each mouse and displayed in FIG. 6B. Both PIO and KDT501 treated DIO female mice exhibited improved insulin sensitivity relative to VEH treated DIO females.

### Both KDT501 and PIO Attenuate Fatty Liver in DIO Female Mice

Liver tissues were collected and placed immediately in 10% formalin. 5 μm sections of tissue were stained with H&E by the Johns Hopkins Molecular and Comparative Pathobiology Phenotyping Core. Quantification of the area of fat in liver tissue was measured with NIH imageJ software and compared across groups.

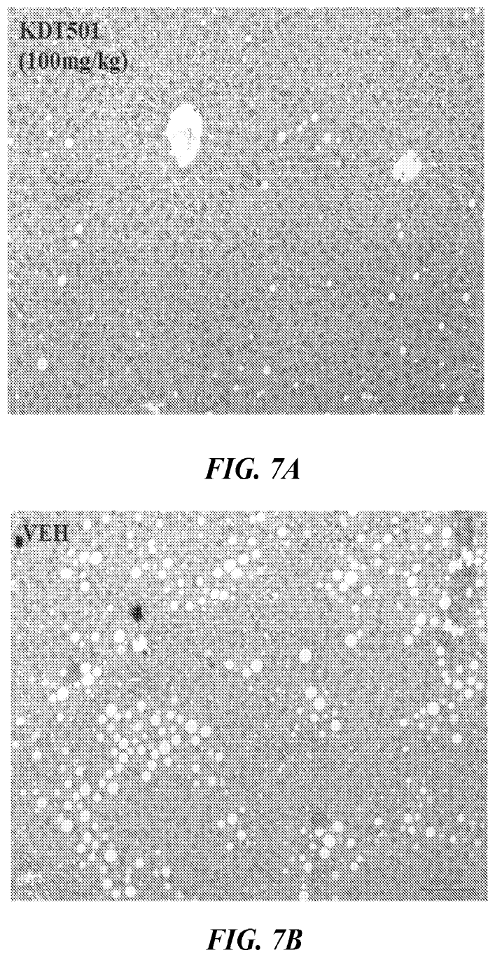

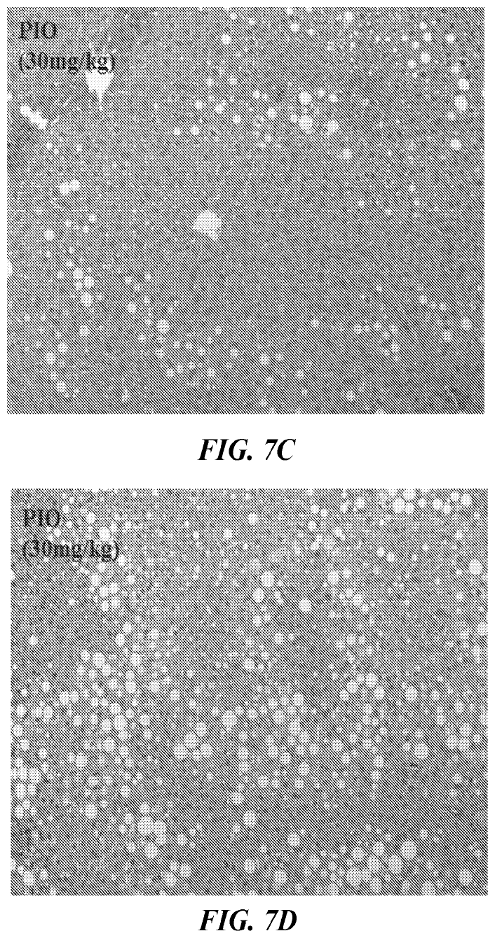

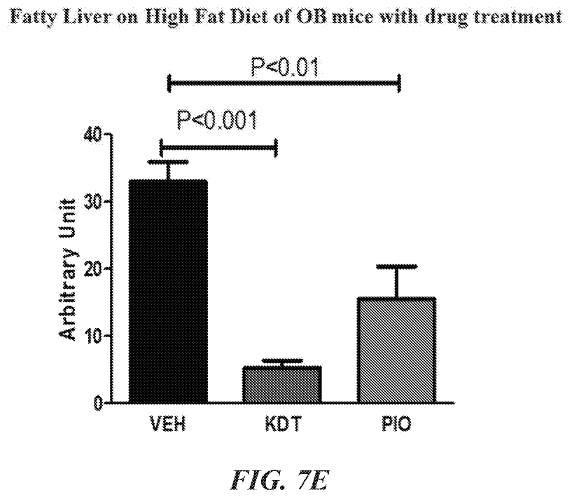

H & E staining of livers clearly revealed lipid accumulation in VEH treated DIO female mice (FIG. 7B). Mice treated with KDT501 had significantly less lipid accumulation than in PIO or VEH treated mice (FIG. 7A and quantified in FIG. 7E). There was a significantly lower amount of lipid deposition in PIO treated mice than in VEH treated mice (FIGS. 7C-7E).

### Metabolic Hormone Profile Not Impacted by PIO or KDT501

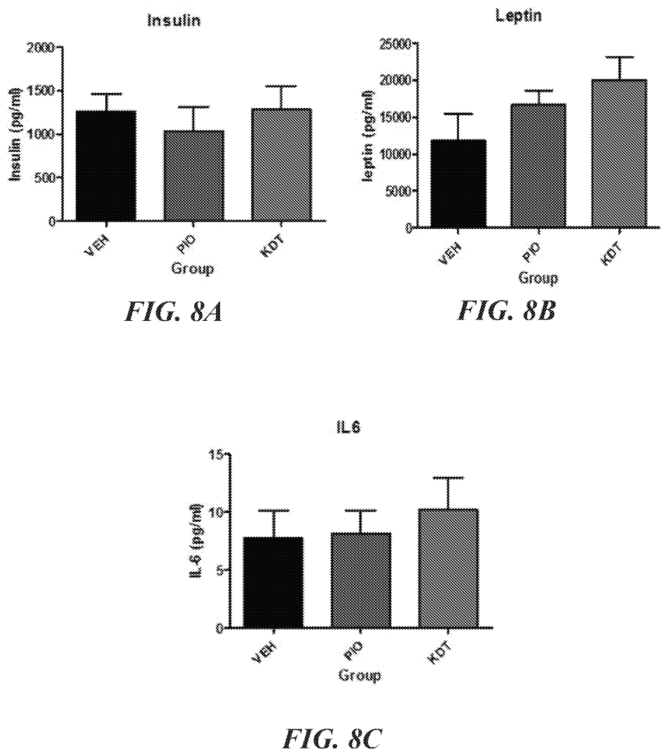

Fasting serum insulin, leptin and IL6 levels were measured by Luminex assay and were not significantly different in DIO female mice treated with PIO, KDT501 or VEH (FIG. 8). Given that there was no change observed in LH and FSH levels (FIG. 2), this suggests that improved ovulatory capacity may result from improved metabolic function resulting from KDT or PIO treatment.

Together these studies sought to determine if KDT501 could contribute to improved metabolic status in DIO female mice and concurrently ameliorate reproductive dysfunction. While control mice in this study gained weight (FIG. 1), it was not to the magnitude previously observed (Brothers et al. 2010, Wu et al. 2012, Wu et al. 2014) suggesting that the oral gavage procedure used to deliver drug impacted feeding behavior. It is contemplated that this could be due to the volume of the drug filling the stomach (not likely since the volume was only 150 μl), or possibly to chronic low level stress axis activation which has been shown to reduce weight gain in DIO rodent models (Harris et al. 1998). This could possibly explain why DIO females treated with PIO did not exhibit increased weight gain as observed in males (Konda et al. 2014). Whether this difference is a sex-specific difference in the effects of stress, or a more general sex difference in drug effects is not clear. However, treatment of female mice with KDT501 resulted in a significant attenuation of weight gain in females fed HFD relative to VEH treated controls. The effects of KDT501 on weight in females closely resembles the effects observed in males (Konda et al. 2014), suggesting a sex difference in the pharmacology of PIO.

Further evidence for sex specific differences in the pharmacological impact of PIO is the dramatic difference in hepatic steatosis in DIO male mice versus DIO female mice. In the former, there is a significant increase in lipid content in the liver in response to PIO treatment relative to VEH treatment. However, in females, PIO significantly reduced lipid accumulation in the liver relative to VEH treatment (FIG. 7). As for the effects on weight, KDT501 had similar hepatic lipid reducing effects in both males (This would be a reference from the Male paper) and females (FIG. 7).

One of the salient features of the female DIO model of infertility was the high LH secretion induced by insulin action at the level of the GnRH neuron and the pituitary (Brothers et al. 2010, DiVall et al. 2015). Given that there was no change observed in LH and FSH levels (FIG. 2), this suggests that improved ovulatory capacity resulted from direct ovarian response to improved metabolic function resulting from KDT or PIO treatment. Similarly, improved estrous cyclicity (FIG. 4) in KDT treated DIO females may be due to improved endocrine function of the ovary.

These studies document a marked improvement in metabolic function in DIO female mice following treatment with KDT501. While PIO treatment was also observed to improve glucose tolerance and insulin sensitivity, surprisingly it was not as efficacious as KDT501 in this model, and additionally was far less effective in ameliorating lipid accumulation in the liver. Both PIO and KDT501 were also shown to improve reproductive cyclicity and ovulatory function in DIO female mice, although the results for PIO were less consistent and thus did not reach statistical significance for either of these endpoints. These studies suggest that KDT501 could represent a novel therapeutic option for women struggling with the metabolic and reproductive health dysfunction associated with PCOS. These studies further suggest that the effect of KDT501 in the treatment of PCOS differs from that of current therapies, including metformin, thiazolidenediones such as PIO, hormones, and GnRH receptor antagonists and further support a different mechanism of action for KDT501 compared to these classes of compounds. It is contemplated that KDT501 activity may be mediated, at least in part, by interactions with bitter taste receptors.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Blank et al. Semin Reprod Med 25(5): 352-359 (2007)
2. Brothers et al. Cell Metab 12(3): 295-305 (2010)

### 3. Brun & Spiegelman J Endocrinol 155(2): 217-218 (1997)

4. Corbould et al. Am J Physiol Endocrinol Metab 288(5): E1047-E1054 (2005)
5. Desai et al. Inflamm Res 58(5): 229-234 (2009)
6. Dimitriadis et al. Curr Pharm Design 22(36): 5535-5546 (2016)
7. DiVall et al. PLoS One 10(3): e 0119995 (2015)

### 8. Dunaif Endocr Rev 18(6): 774-800 (1997)

9. Ehrmann et al. Endocr Rev 16(3): 322-353 (1995)
10. Everard et al. PLoS One 7(3): e33858 (2012)
11. Hall et al. Phytochemistry 69(7): 1534-1547 (2008)
12. Harris et al. Am J Physio! 275(6 Pt 2): R1928-R1938 (1998)
13. Helena et al. J Endocrinol 188(2): 155-165 (2006)
14. Kiefer et al. Diabetes 59(4): 935-946 (2010)
15. Konda et al. Arthritis Rheum 62(6): 1683-1692 (2010)
16. Konda et al. PLoS One 9(1): e87848 (2014)
17. Legro et al. J Clin Endocrinol Metab 98(12): 4565-4592 (2013)

### 18. Lehrke & Lazar Cell 123(6): 993-999 (2005)

19. Lizneva et al. Fertil Steril 106(1): 6-15 (2016)
20. Nelson et al. Biol Reprod 27(2): 327-339 (1982)
21. Nelson et al. Mol Endocrinol 13(6): 946-957 (1999)
22. Norman et al. Lancet 370(9588): 685-697 (2007)
23. Orio et al. Clin Endocrinol (Oxf) 85(5): 764-771 (2016)
24. Pastor et al. J Clin Endocrinol Metab 83(2): 582-590 (1998)
25. Peraza et al. Toxicol Sci 90(2): 269-295 (2006)
26. Semple et al. J Clin Invest 116(3): 581-589 (2006)
27. Shimizu et al. Diabetes Care 21(9): 1470-1474 (1998)
28. Tortoriello et al. Endocrinology 145(3): 1238-1247 (2004)
29. Tortoriello et al. Int J Obes (Lond) 31(3): 395-402 (2007)
30. Tripp et al. Acta Hort (ISHS) 848: 221-234 (2009)
31. Urban et al. Angew Chem Int Ed Engl 52(5): 1553-1555 (2013)
32. Vause et al. J Obstet Gynaecol Can 32(5): 495-502 (2010)
33. Vroegrijk et al. Nutrition 29(1): 276-283 (2013)
34. Wu et al. Diabetes 63(4): 1270-1282 (2014)
35. Wu et al. Diabetes 61(1): 114-123 (2012)
36. Yajima et al. J Biol Chem 279(32): 33456-33462 (2004)
37. Yilmaz et al. J Endocrinol Invest 28(11): 1003-1008 (2005)

1. A method for treating polycystic ovary syndrome (PCOS) in a mammal in need thereof, the method comprising administering to the mammal a therapeutically effective amount of a tetrahydro-iso-alpha acid (THIAA) derivative and/or salt thereof.

2. The method of claim 1, wherein the PCOS is non-insulin resistant PCOS.

3. The method of claim 1, wherein the mammal is administered a chalcone with the THIAA derivative and/or salt thereof.

4. The method of claim 1, wherein the mammal is administered metformin with the THIAA derivative and/or salt thereof.

5. The method of claim 1, wherein the treating comprises preventing or delaying the development of one or more symptoms associated with PCOS.

6. The method of claim 1, wherein the treating comprises reducing or eliminating one or more symptoms associated with PCOS.

7. The method of claim 5, wherein the one or more symptoms are selected from the group consisting of increased insulin levels, increased insulin sensitivity, increased blood glucose, increased serum lipid levels, increased testosterone levels, impaired fertility, amenorrhea, oligomenorrhea, and anovulation.

8. The method of claim 7, wherein the serum lipid levels are cholesterol and/or triglycerides.

9. The method of claim 7, wherein the impaired fertility is detected by measuring at least one marker of fertility.

10. The method of claim 9, wherein the at least one marker of fertility is cyclicity and/or ovulation.

11. The method of claim 1, wherein the THIAA derivative and/or salt thereof has a direct effect on ovarian function.

12. The method of claim 1, wherein the mammal exhibits a decrease in liver fat content.

13. The method of claim 1, wherein the THIAA derivative and/or salt thereof comprises KDT500, KDT501, or a combination thereof.

14. The method of claim 13, wherein the KDT501 is administered as an enantiomerically pure composition of (+) KDT501.

15. The method of claim 1, wherein the THIAA derivative and/or salt thereof is administered to the mammal once a day.

16. The method of claim 1, wherein the THIAA derivative and/or salt thereof is administered to the mammal two or more times per day.

17. The method of claim 1, wherein THIAA derivative and/or salt thereof is a synthetic derivative of a THIAA derivative and/or salt thereof

18. The method of claim 6, wherein the one or more symptoms are selected from the group consisting of increased insulin levels, increased insulin sensitivity, increased blood glucose, increased serum lipid levels, increased testosterone levels, impaired fertility, amenorrhea, oligomenorrhea, and anovulation.
